(19) United States
(12) Patent Application Publication
(10) Pub. No.: US 2007/0082873 A1
(43) Pub. Date: Apr. 12, 2007

(54) Combinations comprising ampa receptor antagonists for the treatment of schizophrenia

(76) Inventors: Kurt Lingenhöhl, Weil am Rhein (DE); Ference Martenyi, Budapest (HU); Silvio Ofner, Muenchenstein (CH); Mary Ann Karolchyk, Morris Township, NJ (US); Hans O. Kalkman, Basel (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/577,196

(22) PCT Filed: Oct. 27, 2004
(86) PCT No.: PCT/EP04/12150

§ 371 Date: Jun. 21, 2006

(30) Foreign Application Priority Data
Oct. 28, 2003 (GB) 0325175.8

(51) Int. Cl. A61K 31/675

(52) U.S. Cl. 514/80

(57) ABSTRACT

The present invention relates to combinations suitable for the treatment of psychiatric/neurological disorders, in particular schizophrenia. The combinations comprise at least one AMPA receptor antagonist and at least one compound selected from the group consisting of (a) anti-epileptic drugs selected from barbiturates and derivatives thereof, benzodiazepines, carboxamides, hydantoins, succinimides, valproic acid and other fatty acid derivates and other anti-epileptic drugs, (b) conventional antipsychotics and (c) atypical antipsychotics.

The present invention relates to combinations suitable for the treatment of psychiatric/neurological disorders, in particular schizophrenia.

Surprisingly, it has been found that the effect of a combination which comprises at least one AMPA receptor antagonist and at least one compound selected from the group consisting of (a) anti-epileptic drugs selected from barbiturates and derivatives thereof, benzodiazepines, carboxamides, hydantoins, succinimides, valproic acid and other fatty acid derivates and other anti-epileptic drugs, (b) conventional antipsychotics and (c) atypical antipsychotics is greater than the additive effect of the combined drugs. Furthermore, the combinations disclosed herein can be used to treat schizophrenia which is refractory to monotherapy employing one of the combination partners alone.

Hence, the invention relates to a combination, such as a combined preparation or pharmaceutical composition, which comprises at least one AMPA receptor antagonist and at least one compound selected from the group consisting of (a) anti-epileptic drugs selected from barbiturates and derivatives thereof, benzodiazepines, carboxamides, hydantoins, succinimides, valproic acid and other fatty acid derivates and other anti-epileptic drugs, (b) conventional antipsychotics and (c) atypical antipsychotics, in which the active ingredients are present in each case in free form or in the form of a pharmaceutically acceptable salt and optionally at least one pharmaceutically acceptable carrier; for simultaneous, separate or sequential use.

The term “AMPA receptor antagonists” as used herein includes, but is not limited to the quinoxaline-dione aminoalkylphosphonates of formula I 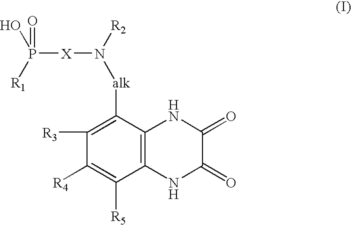
wherein
R₁ represents hydroxy or an aliphatic, aryl aliphatic or aromatic group,
X represents an aliphatic, cycloaliphatic, cycloaliphatic aliphatic, aryl aliphatic, heteroaryl aliphatic or aromatic group,
R₂ represents hydrogen or an aliphatic or aryl aliphatic group, alk stands for C1-C7alkylene, and
R₃, R₄ and R₅ represent independently of each other hydrogen, C1-C7alkyl, halogen, trifluoromethyl, cyano or nitro,
and wherein the radicals have the meanings as defined in WO 98/17672.

AMPA receptor antagonists include also, EGIS 8332 (7-acetyl-5-(4-aminophenyl)-8,9-dihydro-8-methyl-7H-1,3-dioxolo[4,5-h][2,3]benzodiazepine-8-carbonitrile), GYKI 47261 (4-(7-chloro-2-methyl-4H-3,10,10a-triaza-benzo[f]azulen-9-yl)-phenylamine), irampanel (BIIR 561; N,N-dimethyl-2-[2-(3-phenyl-1,2,4-oxadiazol-5-yl)phenoxy]ethanamine), KRP 199 (7-[4-[[[[(4-carboxyphenyl)-amino]carbonyl]oxy]methyl]-1H-imidazol-1-yl]-3,4-dihydro-3-oxo-6-(trifluoromethyl)-2-quinoxalinecarboxylic acid), NS 1209 (2-[[[5-[4-[(dimethylamino)-sulfonyl]-phenyl]-1,2,6,7,8,9-hexahydro-8-methyl-2-oxo-3H-pyrrolo[3,2-h]isoquinolin-3-ylidene]amino]oxy]-4-hydroxybutanoic acid monosodium salt, e.g. prepared as described in WO 98/14447), topiramate (TOPAMAX, 2,3:4,5-bis-O-(1-methylethylidene)-beta-D-fructo-pyranose sulfamate, preparation, e.g. as described in U.S. Pat. No. 535,475), talampanel (LY-300164, (R)-7-acetyl-5-(4-aminophenyl)-8,9-dihydro-8-methyl-7H-1,3-dioxolo[4,5-h][2,3]benzodiazepine, preparation, e.g. as described in EP 492485), YM90K (6-imidazol-1-yl-7-nitro-1,4-dihydro-quinoxaline-2,3-dione), S-34730 (7-chloro-6-sulfamoyl-2-(1H)-quinolinone-3-phosphonic acid), Zonampanel (YM-872; (7-imidazol-1-yl-6-nitro-2,3-dioxo-3,4-dihydro-2H-quinoxalin-1-yl)-acetic acid), GYKI-52466 (4-(8-methyl-9H-1,3-dioxa-6,7-diazacyclohepta[f]inden-5-yl)-phenylamine), ZK-200775 (MPQX, (7-morpholin-4-yl-2,3-dioxo-6-trifluoromethyl-3,4-dihydro-2H-quinoxalin-1-ylmethyl)-phosphonic acid), CP465022 (3-(2-chloro-phenyl)-2-[2-(6-diethylaminomethyl-pyridin-2-yl)-vinyl]-6-fluoro-3H-quinazolin-4-one), SYM-2189 (4-(4-amino-phenyl)-6-methoxy-1-methyl-1H-phthalazine-2-carboxylic acid propylamide), SYM-2206 (8-(4-amino-phenyl)-5-methyl-5H-[1,3]dioxolo[4,5-g]phthalazine-6-carboxylic acid propylamide, RPR-117824 ((4-oxo-2-phosphono-5,10-dihydro-4H-imidazo[1,2-a]indeno[1,2-e]pyrazin-9-yl)-acetic acid), LY-293558 (6-[2-(1H-tetrazol-5-yl)-ethyl]-decahydro-isoquinoline-3-carboxylic acid).

The term “barbiturates and derivatives thereof” as used herein includes, but is not limited to phenobarbital, pentobarbital, mepobarbital and primidon. The term “benzodiazepines” as used herein includes, but is not limited to clonazepam, diazepam and lorazepam. The term “carboxamides” as used herein includes, but is not limited to carbamazepine, oxcarbazepine, 10-hydroxy-10,11-dihydrocarbamazepine and the compounds of formula II 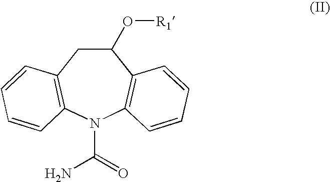
wherein R₁′ represents C₁-C₃alkyl carbonyl. The term “hydantoins” as used herein includes, but is not limited to phenytoin. The term “succinimides” as used herein includes, but is not limited to ethosuximide, phensuximide and mesuximide. The term “valproic acid and other fatty acid derivates” as used herein includes, but is not limited to valproic acid sodium salt, tiagabine hydrochloride monohydrate and vigrabatrine. The term “other anti-epileptic drugs” as used herein includes, but is not limited to levetiracetam, lamotrigine, gabapentin, sultiam, felbamate, the 1,2,3-1H-triazoles disclosed in EP 114 347 and the 2-aryl-8-oxodihydropurines disclosed in WO99/28320.

The term “conventional antipsychotics” as used herein includes, but is not limited to haloperidol, fluphenazine, thiotixene and flupentixol.

The term “atypical antipsychotics” as used herein relates to clozaril, risperidone, olanzapine, quetiapine, ziprasidone and aripiprazol.

The structure of the active ingredients identified by code nos., generic or trade names and their preparation may be taken from the actual edition of the standard compendium “The Merck Index” (e.g. M. J. O'Neil et al., ed., ‘The Merck Index’, 13^{th} ed., Merck Research Laboratories, 2001) or from databases, e.g. Patents International (e.g. IMS World Publications). The corresponding content thereof is hereby incorporated by reference. Any person skilled in the art is fully enabled to identify the active ingredients and, based on these references, likewise enabled to manufacture and test the pharmaceutical indications and properties in standard test models, both in vitro and in vivo.

Phenobarbital, can be administered, e.g., in the form as marketed, e.g. under the trademark Luminal™. Primidon can be administered, e.g., in the form as marketed, e.g. under the trademark Mylepsinum™. Clonazepam can be administered, e.g., in the form as marketed, e.g. under the trademark Antelepsin™. Diazepam can be administered, e.g., in the form as marketed, e.g. under the trademark Diazepam Desitin™. Lorazepam can be administered, e.g., in the form as marketed, e.g. under the trademark Tavor™. Carbamazepine can be administered, e.g., in the form as marketed, e.g. under the trademark Tegretal™ or Tegretol™. Oxcarbazepine can be administered, e.g., in the form as marketed, e.g. under the trademark Trileptal™. Oxcarbazepine is well known from the literature [see for example Schuetz H. et al., Xenobiotica (GB), 16(8), 769-778 (1986)]. The preparation of the compound of formula II wherein R₁′ is C₁-C₃alkyl carbonyl and its pharmaceutically acceptable salts is described, e.g., in U.S. Pat. No. 5,753,646. 10-Hydroxy-10,11-dihydrocarbamazepine can be prepared as disclosed in U.S. Pat. No. 3,637,661. 10-Hydroxy-10,11-dihydrocarbamazepine may be administered, e.g., in the form as described in U.S. Pat. No. 6,316,417. Phenytoin can be administered, e.g., in the form as marketed, e.g. under the trademark Epanutin™. Ethosuximide can be administered, e.g., in the form as marketed, e.g. under the trademark Suxinutin™. Mesuximide can be administered, e.g., in the form as marketed, e.g. under the trademark Petinutin™. Valproic acid sodium salt can be administered, e.g., in the form as marketed, e.g. under the trademark Leptilan™. Tiagabine hydrochloride monohydrate can be administered, e.g., in the form as marketed, e.g. under the trademark Gabitril™. Vigabatrine can be administered, e.g., in the form as marketed, e.g. under the trademark Sabril™. Levetiracetam can be administered, e.g., in the form as marketed, e.g. under the trademark Keppra™. Lamotrigine can be administered, e.g., in the form as marketed, e.g. under the trademark Lamictal™. Gabapentin can be administered, e.g., in the form as marketed, e.g. under the trademark Neurontin™. Sultiam can be administered, e.g., in the form as marketed, e.g. under the trademark Ospolot™. Felbamate can be administered, e.g., in the form as marketed, e.g. under the trademark Taloxa™. Topiramate can be administered, e.g., in the form as marketed, e.g. under the trademark Topamax™. The 1,2,3-1H-triazoles disclosed in EP 114 347 may be administered, e.g., in the form as described in U.S. Pat. No. 6,455,556. The 2-aryl-8-oxodihydropurines disclosed in WO99/28320 may be administered, e.g., in the form as described in WO99/28320. Haloperidol can be administered, e.g., in the form as marketed, e.g. under the trademark Haloperidol STADA™. Fluphenazine can be administered, e.g., in the form of its dihydrochloride as marketed, e.g. under the trademark Prolixin™. Thiothixene can be administered, e.g., in the form as marketed, e.g. under the trademark Navane™. It can be prepared, e.g., as described in U.S. Pat. No. 3,310,553. Flupentixol can be administered for instance in the form of its dihydrochloride, e.g., in the form as marketed, e.g. under the trademark Emergil™ or in the form of its decanoate, e.g., in the form as marketed, e.g. under the trademark Depixol™. It can be prepared, e.g., as described in BP 925,538. Clozaril can be administered, e.g., in the form as marketed, e.g. under the trademark Leponex™. It can be prepared, e.g., as described in U.S. Pat. No. 3,539,573. Risperidone can be administered, e.g., in the form as marketed, e.g. under the trademark Risperdal™. Olanzapine can be administered, e.g., in the form as marketed, e.g. under the trademark Zyprexa™. Quetiapine can be administered, e.g., in the form as marketed, e.g. under the trademark Seroquel™. Ziprasidone can be administered, e.g., in the form as marketed, e.g. under the trademark Geodon™. It can be prepared, e.g., as described in GB 281,309. Aripiprazole can be administered, e.g., in the form as marketed, e.g. under the trademark Abilify™. It can be prepared, e.g., as described in U.S. Pat. No. 5,006,528. Topiramate can be administered, e.g., in the form as marketed, e.g. under the trademark Topamax™. The compounds of formula I as well as their production process and pharmaceutical compositions thereof are known e.g. from WO 98/17672.

The term “a combined preparation”, as used herein defines especially a “kit of parts” in the sense that the first and second active ingredient as defined above can be dosed independently or by use of different fixed combinations with distinguished amounts of the ingredients, i.e., simultaneously or at different time points. The parts of the kit of parts can then, e.g., be administered simultaneously or chronologically staggered, that is at different time points and with equal or different time intervals for any part of the kit of parts. Very preferably, the time intervals are chosen such that the effect on the treated disease in the combined use of the parts is larger than the effect which would be obtained by use of only any one of the active ingredients. The ratio of the total amounts of the active ingredient 1 to the active ingredient 2 to be administered in the combined preparation can be varied, e.g., in order to cope with the needs of a patient sub-population to be treated or the needs of the single patient which different needs can be due to age, sex, body weight, etc. of the patients. Preferably, there is at least one beneficial effect, e.g., a mutual enhancing of the effect of the first and second active ingredient, in particular a synergism, e.g. a more than additive effect, additional advantageous effects, less side effects, a combined therapeutically effect in a non-effective dosage of one or both of the first and second active ingredient, and especially a strong synergism the first and second active ingredient.

It will be understood that in the discussion of methods, references to the active ingredients are meant to also include the pharmaceutically acceptable salts. If these active ingredients have, for example, at least one basic center, they can form acid addition salts. Corresponding acid addition salts can also be formed having, if desired, an additionally present basic center. The active ingredients having an acid group (for example COOH) can also form salts with bases. The active ingredient or a pharmaceutically acceptable salt thereof may also be used in form of a hydrate or include other solvents used for crystallization.

A pharmaceutical combination which comprises at least one AMPA antagonist and at least one compound selected from the group consisting of (a) anti-epileptic drugs selected from barbiturates and derivatives thereof, benzodiazepines, carboxamides, hydantoins, succinimides, valproic acid and other fatty acid derivates and other anti-epileptic drugs, (b) conventional antipsychotics and (c) atypical antipsychotics, in which the active ingredients are present in each case in free form or in the form of a pharmaceutically acceptable salt, if at least one salt-forming group is present, will be referred to hereinafter as a COMBINATION OF THE INVENTION.

Surprisingly it was found that the administration of a COMBINATION OF THE INVENTION results in a beneficial, especially a synergistic, therapeutic effect or in other surprising beneficial effects, e.g. less side effects, compared to a monotherapy applying only one of the pharmaceutically active ingredients used in the COMBINATION OF THE INVENTION. The antipsychotic potential of a COMBINATION OF THE INVENTION may, for example, be evidenced in preclinical studies known as such, e.g. the methods described herein.

The antipsychotic potential of the COMBINATION OF THE INVENTION is indicated in standard tests, e.g. in the amphetamine-induced hyperlocomotion test. Blockade of amphetamine-induced hyperlocomotion is well known as screening paradigm for antischizophrenic activity.

Male rats are used. In principle 4 treatment groups are formed:
1) AMPA receptor antagonist followed by solvent 2 and solvent 3 to study the effects of the AMPA receptor antagonist on locomotor activity.
2) Solvent 1, combination partner and solvent 3 to study the effects of the combination partner on locomotor activity.
3) Solvent 1, solvent 2, followed by amphetamine to study the induction of hyperlocomotor activity.
4) AMPA receptor antagonist followed by solvent 2 and amphetamine.
5) Solvent 1 followed by combination partner and amphetamine.
6) The COMBINATION OF THE INVENTION (doses of each active ingredients at doses close to threshold) followed by solvent 3.
7) The COMBINATION OF THE INVENTION (doses of each active ingredients at doses close to threshold) followed by amphetamine.
8) Solvent 1-solvent 2-solvent 3.

Rats are randomly allocated to these pretreatment groups (n=10/dose group). Drugs are administered subcutaneous (s.c.), 15 min prior to SDZ 220-581. Immediately after the animals received amphetamine, they are placed into the activity monitor for a period of 60 min. Locomotor activity is analysed over the initial 30 minutes.

Threshold doses of each active ingredient of the combination partners are used. Amphetamine is dosed at 1 mg/kg s.c. Locomotion is recorded with a videotracking system. Animals are on a normal 12/12 h. day-night cycle, with light on at 06:00 H. Experiments are performed in a dimly lit room between 07:00 H and 15:00 H. Animals are placed in a round arena (diameter 42 cm, height 32 cm) made of grey polyvinylchloride plastic. The camera is placed such, that four animals (one per arena) can be recorded simultaneously.

Comparison between groups is done with Student's t-test, corrected for multiple testing using the Bonferroni procedure.

Furthermore, the pharmacological activity of a COMBINATION OF THE INVENTION may, for example, be demonstrated in a clinical study. Such clinical studies are preferably randomized, double-blind, clinical studies in patients with schizophrenia. Such studies demonstrate, in particular, the synergism of the active ingredients of the COMBINATIONS OF THE INVENTION. The beneficial effects on schizophrenia can be determined directly through the results of these studies or by changes in the study design which are known as such to a person skilled in the art. The studies are, in particular, suitable to compare the effects of a monotherapy using the active ingredients and a COMBINATION OF THE INVENTION.

The COMBINATIONs OF THE INVENTION provide, in particular, benefits in the treatment of positive symptoms, negative symptoms, mood symptoms and/or cognitive symptoms of schizophrenia and/or psychosis. Furthermore, some of the COMBINATIONs OF THE INVENTION show beneficial effects in the control of impulsive and/or violent behavior of schizophrenic patients.

A further benefit is that lower doses of the active ingredients of the COMBINATION OF THE INVENTION can be used, for example, that the dosages need not only often be smaller but are also applied less frequently, or can be used in order to diminish the incidence of side effects. This is in accordance with the desires and requirements of the patients to be treated. The COMBINATIONs OF THE INVENTION can be used, in particular, for the treatment of schizophrenia which is refractory to monotherapy.

In one embodiment of the invention, the AMPA receptor antagonists used in the present invention are competitive AMPA receptor antagonists.

Preferably, the COMBINATION OF THE INVENTION comprises an AMPA receptor antagonist, which is a quinoxalinedione aminoalkylphosphonate, in particular a quinoxalinedione aminoalkylphosphonate of formula I 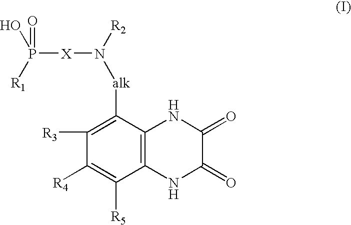
wherein
R₁ represents hydroxy or an aliphatic, aryl aliphatic or aromatic group,
X represents an aliphatic, cycloaliphatic, cycloaliphatic aliphatic, aryl aliphatic, heteroaryl aliphatic or aromatic group,
R₂ represents hydrogen or an aliphatic or aryl aliphatic group, alk stands for C1-C7alkylene, and
R₃, R₄ and R₅ represent independently of each other hydrogen, C1-C7alkyl, halogen, trifluoromethyl, cyano or nitro,
and wherein the radicals have the meanings as defined in WO 98/17672.

Most preferably, the COMBINATION OF THE INVENTION comprises an AMPA receptor antagonist which is a compound of formula I, wherein R₁ represents hydroxyl, X represents methylene, R₂ represents hydrogen, alk stands for methylene, R₃ and R₅ represent hydrogen, and R₄ represents nitro or a salt thereof, i.e. is {[(7-Nitro-2,3-dioxo-1,2,3,4-tetrahydro-quinoxalin-5-ylmethyl)-amino]-methyl}-phosphonic acid or a salt thereof (cf. WO 97/08155, Example 57, 1st entry, and WO 98/17672, Example 12, 4th entry).

The disclosures of WO 97/08155 and WO 98/17672 are incorporated by reference.

In another embodiment of the present invention, the AMPA receptor antagonists is selected from EGIS 8332 (7-acetyl-5-(4-aminophenyl)-8,9-dihydro-8-methyl-7H-1,3-dioxolo[4,5-h][2,3]benzodiazepine-8-carbonitrile), GYKI 47261 4-(7-chloro-2-methyl-4H-3,10,10a-triaza-benzo[f]azulen-9-yl)-phenylamine), irampanel (BIIR 561; N,N-dimethyl-2-[2-(3-phenyl-1,2,4-oxadiazol-5-yl)phenoxy]ethanamine), KRP 199 (7-[4-[[[[(4-carboxyphenyl)amino]carbonyl]oxy]methyl]-1H-imidazol-1-yl]-3,4-dihydro-3-oxo-6-(trifluoromethyl)-2-quinoxalinecarboxylic acid), NS 1209 (2-[[[5-[4-[(dimethylamino)-sulfonyl]phenyl]-1,2,6,7,8,9-hexahydro-8-methyl-2-oxo-3H-pyrrolo[3,2-h]isoquinolin-3-ylidene]amino]oxy]-4-hydroxybutanoic acid monosodium salt, e.g. prepared as described in WO 98/14447), topiramate (TOPAMAX, 2,3:4,5-bis-O-(1-methylethylidene)-beta-D-fructopyranose sulfamate, preparation, e.g. as described in U.S. Pat. No. 535,475), talampanel (LY-300164, (R)-7-acetyl-5-(4-aminophenyl)-8,9-dihydro-8-methyl-7H-1,3-dioxolo[4,5-h][2,3]benzo-diazepine, preparation, e.g. as described in EP 492485), YM90K (6-imidazol-1-yl-7-nitro-1,4-dihydro-quinoxaline-2,3-dione), S-34730 (7-chloro-6-sulfamoyl-2-(1H)-quinolinone-3-phosphonic acid), Zonampanel (YM-872; (7-imidazol-1-yl-6-nitro-2,3-dioxo-3,4-dihydro-2H-quinoxalin-1-yl)-acetic acid), GYKI-52466 (4-(8-methyl-9H-1,3-dioxa-6,7-diaza-cyclohepta[f]inden-5-yl)-phenylamine), ZK-200775 (MPQX, (7-morpholin-4-yl-2,3-dioxo-6-trifluoromethyl-3,4-dihydro-2H-quinoxalin-1-ylmethyl)-phosphonic acid), CP465022 (3-(2-chloro-phenyl)-2-[2-(6-diethylaminomethyl-pyridin-2-yl)-vinyl]-6-fluoro-3H-quinazolin4-one), SYM-2189 (4-(4-amino-phenyl)-6-methoxy-1-methyl-1H-phthalazine-2-carboxylic acid propylamide), SYM-2206 (8-(4-amino-phenyl)-5-methyl-5H-[1,3]dioxolo[4,5-g]phthalazine-6-carboxylic acid propylamide, RPR-117824 ((4-oxo-2-phosphono-5,10-dihydro-4H-imidazo[1,2-a]indeno[1,2-e]pyrazin-9-yl)-acetic acid), LY-293558 (6-[2-(1H-tetrazol-5-yl)-ethyl]-decahydro-isoquinoline-3-carboxylic acid).

It is one objective of this invention to provide a pharmaceutical composition comprising a quantity, which is jointly therapeutically effective against schizophrenia, comprises at least one AMPA antagonist, at least one compound selected from the group specified above and at least one pharmaceutically acceptable carrier. In this composition, the first and second active ingredient can be administered together, one after the other or separately in one combined unit dosage form or in two separate unit dosage forms. The unit dosage form may also be a fixed combination.

The pharmaceutical compositions according to the invention can be prepared in a manner known per se and are those suitable for enteral, such as oral or rectal, and parenteral administration to mammals (warm-blooded animals), including humans, comprising a therapeutically effective amount of at least one pharmacologically active ingredient, alone or in combination with one or more pharmaceutically acceptable carries, especially suitable for enteral or parenteral application. The preferred route of administration of the dosage forms of the present invention is orally.

The novel pharmaceutical composition contain, for example, from about 10% to about 100%, preferably from about 20% to about 60%, of the active ingredients. Pharmaceutical preparations for the combination therapy for enteral or parenteral administration are, for example, those in unit dosage forms, such as sugar-coated tablets, tablets, capsules or suppositories, and furthermore ampoules. If not indicated otherwise, these are prepared in a manner known per se, for example by means of conventional mixing, granulating, sugar-coating, dissolving or lyophilizing processes. It will be appreciated that the unit content of active ingredient or ingredients contained in an individual dose of each dosage form need not in itself constitute an effective amount since the necessary effective amount can be reached by administration of a plurality of dosage units.

In preparing the compositions for oral dosage form, any of the usual pharmaceutical media may be employed, such as, for example, water, glycols, oils or alcohols; or carriers such as starches, sugars, microcristalline cellulose, diluents, granulating agents, lubricants, binders, disintegrating agents and the like in the case of oral solid preparations such as, for example, powders, capsules and tablets. Because of their ease of administration, tablets and capsules represent the most advantageous oral dosage unit form in which case solid pharmaceutical carriers are obviously employed.

Furthermore, the present invention relates to the use of a COMBINATION OF THE INVENTION for the preparation of a medicament for the treatment of schizophrenia.

Additionally, the present invention provides a method of treating a warm-blooded animal having schizophrenia comprising administering to the animal a COMBINATION OF THE INVENTION in a quantity which is jointly therapeutically effective against schizophrenia and in which the compounds can also be present in the form of their pharmaceutically acceptable salts.

Moreover, the present invention provides a commercial package comprising as active ingredients COMBINATION OF THE INVENTION, together with instructions for simultaneous, separate or sequential use thereof in the treatment of schizophrenia.

In particular, a therapeutically effective amount of each of the active ingredients of the COMBINATION OF THE INVENTION may be administered simultaneously or sequentially and in any order, and the components may be administered separately or as a fixed combination. For example, the method of treatment of schizophrenia according to the invention may comprise (i) administration of the first active ingredient in free or pharmaceutically acceptable salt form and (ii) administration of the second active ingredient in free or pharmaceutically acceptable salt form, simultaneously or sequentially in any order, in jointly therapeutically effective amounts, preferably in synergistically effective amounts, e.g. in daily dosages corresponding to the amounts described herein. The individual active ingredients of the COMBINATION OF THE INVENTION can be administered separately at different times during the course of therapy or concurrently in divided or single combination forms. Furthermore, the term administering also encompasses the use of a pro-drug of an active ingredient that convert in vivo to the active ingredient. The instant invention is therefore to be understood as embracing all such regimes of simultaneous or alternating treatment and the term “administering” is to be interpreted accordingly.

In one preferred embodiment of the invention, the COMBINATION OF THE INVENTION is used for the treatment of treatment of schizophrenia which is refractory to monotherapy.

The effective dosage of each of the active ingredients employed in the COMBINATION OF THE INVENTION may vary depending on the particular compound or pharmaceutical composition employed, the mode of administration, the severity of the condition being treated. Thus, the dosage regimen the COMBINATION OF THE INVENTION is selected in accordance with a variety of factors including the route of administration and the renal and hepatic function of the patient. A physician, clinician or veterinarian of ordinary skill can readily determine and prescribe the effective amount of the single active ingredients required to prevent, counter or arrest the progress of the condition. Optimal precision in achieving concentration of the active ingredients within the range that yields efficacy without toxicity requires a regimen based on the kinetics of the active ingredients' availability to target sites. This involves a consideration of the distribution, equilibrium, and elimination of the active ingredients.

When the combination partners employed in the COMBINATION OF THE INVENTION are applied in the form as marketed as single drugs, their dosage and mode of administration can take place in accordance with the information provided on the packet leaflet of the respective marketed drug in order to result in the beneficial effect described herein, if not mentioned herein otherwise. In particular,

Phenobarbital may be administered to an adult patient in a total daily dosage between about 1 to about 3 mg/kg body weight and to a pediatric patient in a total daily dosage between about 3 to about 4 mg/kg body weight, split into two separate units.

Primidone may be administered to an adult patient and to children being at least 9 years old in a total daily dosage of 0.75 to 1.5 g.

Clonazepam may be administered to an adult patient in a total daily dosage between about 3 to about 8 mg and to a pediatric patient in a total daily dosage between about 0.5 to about 3 mg, split into three of four separate units.

Diazepam may be administered to an adult patient in a total daily dosage between about 5 to about 10 mg and to a pediatric patient in a total daily dosage between about 5 to about 10 mg.

Lorazepam may be administered to an adult patient in a total daily dosage between about 0.044 mg/kg body weight to about 0.05 mg/kg body weight.

Carbamazepine may be administered to an adult patient in a total daily dosage between about 600 to about 2000 mg and to a pediatric patient older than 6 years in a total daily dosage between about 400 to about 600 mg.

Oxcarbazepine may be administered to an adult patient in a total daily dosage between about 600 to about 2400 mg and to a pediatric patient in a total daily dosage between about 30 to about 46 mg/kg body weight.

Phenytoin may be administered to an adult patient in a total daily dosage between about 100 to about 300 mg and to a pediatric patient in a total daily dosage between about 100 to about 200 mg.

Ethosuximide may be administered to an adult patient in a total daily dosage of about 15 mg/kg body weight and to a pediatric patient in a total daily dosage of about 20 mg/kg body weight.

Valproic acid sodium salt may be administered to an adult patient in a total daily dosage of about 20 mg/kg body weight and to a pediatric patient in a total daily dosage of about 30 mg/kg body weight.

Tiagabine hydrochloride monohydrate may be administered to an adult patient in a total daily dosage between about 15 to about 70 mg.

Vigrabatrine may be administered to an adult patient in a total daily dosage between about 2 to about 3 g.

Levetiracetam may be administered to patient who is older than 16 years in a total daily dosage between about 1000 to about 3000 mg.

Lamotrigine may be administered to patient who is older than 12 years in a total daily dosage between about 100 to about 200 mg.

Gabapentin may be administered to patient in a total daily dosage between about 900 to about 2400 mg.

Sultiam may be administered to patient in a total daily dosage between about 5 to about 10 mg/kg body weight.

Felbamate may be administered to patient who is older than 14 years in a total daily dosage of between about 2400 to about 3600 mg.

Topiramate may be administered to an adult patient in a total daily dosage of between about 250 to about 500 mg.

Clozaril may be administered to an adult patient in a total daily dosage of between about 300 to about 900 mg.

Haloperidol may be administered to a patient in a total daily dosage of between about 2.5 to about 30 mg.

Olanzapine can be administered to a patient in a total daily dosage of between about 2.5 to about 20 mg.

Quetiapine can be administered to a patient in a total daily dosage of between about 500 to about 600 mg.

Risperidone may be administered to a patient in a total daily dosage of between about 2 to about 6 mg.

{[(7-Nitro-2,3-dioxo-1,2,3,4-tetrahydro-quinoxalin-5-ylmethyl)-amino]-methyl}-phosphonic acid may be administered to a patient in a total daily dosage of about 60 to about 400 mg.

Talampanel may be administered to a patient in a total daily dosage of between 25 to about 75 mg

1. A combination which comprises at least one AMPA receptor antagonist and at least one compound selected from the group consisting of (a) anti-epileptic drugs selected from barbiturates and derivatives thereof, benzodiazepines, carboxamides, hydantoins, succinimides, valproic acid and other fatty acid derivates and other anti-epileptic drugs, (b) conventional antipsychotics and (c) atypical antipsychotics, in which the active ingredients are present in each case in free form or in the form of a pharmaceutically acceptable salt and optionally at least one pharmaceutically acceptable carrier; for simultaneous, separate or sequential use.

2. Combination according to claim 1 which is a combined preparation or a pharmaceutical composition.

3. Combination according to claim 1 wherein the AMPA receptor antagonist is a compound of formula I 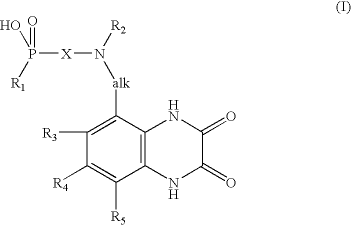
wherein
R1 represents hydroxy or an aliphatic, aryl aliphatic or aromatic group,
X represents an aliphatic, cycloaliphatic, cycloaliphatic aliphatic, aryl aliphatic, heteroaryl aliphatic or aromatic group,
R2 represents hydrogen or an aliphatic or aryl aliphatic group,
alk stands for C1-C7alkylene, and
R3, R4 and R5 represent independently of each other hydrogen, C1-C7alkyl, halogen, trifluoromethyl, cyano or nitro,
or a salt therof.

4. Combination according to claim 3, wherein in the formula I R₁ is hydroxy, R₂ is hydrogen, alk represents methylene, R₃ and R₅ are both hydrogen, R₄ is nitro and X is methylene.

5. Combination according to claim 1 for simultaneous, separate or sequential use in the treatment of schizophrenia.

6. Method of treating a warm-blooded animal having schizophrenia comprising administering to the animal a combination according to claim 1 in a quantity which is jointly therapeutically effective against schizophrenia and in which the compounds can also be present in the form of their pharmaceutically acceptable salts.

7. A pharmaceutical composition comprising a quantity, which is jointly therapeutically effective against schizophrenia, of a pharmaceutical combination according to claim 1 and at least one pharmaceutically acceptable carrier.

8. Use of a combination according to claim 1 for the preparation of a medicament for the treatment of schizophrenia.

9. Use according to claim 5 wherein the schizophrenia is refractory to monotherapy.

10. A commercial package comprising a combination according to claim 1 together with instructions for simultaneous, separate or sequential use thereof in the treatment of schizophrenia.
